(19) United States
(12) Patent Application Publication
(10) Pub. No.: US 2025/0040803 A1
(43) Pub. Date: Feb. 6, 2025

(54) APPARATUS AND METHOD FOR MEASURING PROPERTY OF EYE

(71) Applicant: ICARE FINLAND OY, Vantaa (FI)

(72) Inventors: Mika Salkola, Espoo (FI); Viktor Honkanen, Espoo (FI)

(73) Assignee: ICARE FINLAND OY, Vantaa (FI)

(21) Appl. No.: 18/696,570

(22) PCT Filed: Nov. 7, 2022
(86) PCT No.: PCT/FI2022/050731

§ 371 Date: Mar. 28, 2024

(30) Foreign Application Priority Data
Nov. 19, 2021 (FI) 20216186

(51) Int. Cl. A61B 3/10; A61B 3/16; A61B 8/10

(52) U.S. Cl. CPC A61B 3/1005; A61B 3/16; A61B 8/10

(57) ABSTRACT

Disclosed is an apparatus for measuring property of an eye. The apparatus has an elongated magnetic probe having a probe head, wherein the probe head has an ultrasonic sensor, a driver coil arranged partially to surround the elongated magnetic probe; and a controller configured to, when in use, selectively energize the driver coil to create a magnetic force to initiate movement of the elongated magnetic probe to contact the probe head with a surface of the eye The ultrasonic sensor in the probe head is configured to send an ultrasonic pulse upon contacting the surface of the eye; measure, using the ultrasonic sensor, time taken to receive at least one reflection of plurality of reflections of the ultrasonic pulse, and calculate the property of the eye using value of the time taken to receive at least one reflection of the ultrasonic pulse.

### TECHNICAL FIELD

The present disclosure relates generally to ophthalmic devices; and more specifically, to apparatuses and methods for measuring property of an eye.

### BACKGROUND

Generally, human beings suffer from serious ailments of eye, such as, but not limited to, glaucoma and dry eye syndrome. In order to have an accurate diagnosis of diseases related to the eye, a proper analysis of various properties of the eyes is beneficial. Hence, nowadays, specialized equipment is used to measure physiological properties of the eye, which have been developed over time and are still evolving. For instance, presently, myopia or nearsightedness is a common vision condition in which a person can see objects clearly when they are closer to the eye, but vision gets blurry when the objects are farther away.

Notably, myopia is a common cause of impaired vision in people under the age of 40. Herein, myopia occurs when eyeball is too long, relative to focusing power of cornea and lens of the eye. Consequently, light rays wrongly focus at a point in front of retina of the eye rather than directly on surface of the retina. Typically, myopia begins in childhood, wherein in most cases condition of the eye suffering from myopia stabilizes at some point. However, sometimes myopia continues to progress with age. Practically, there are ways to control progression of myopia in childhood, wherein it is important to monitor changes happening to the eye.

Existing measurement techniques to measure properties of the eye suffer from certain limitations. For instance, the existing measurement techniques require a long measurement time, which may prove to be uncomfortable for a person, especially for a child. Furthermore, the existing measurement techniques use ocular anesthesia, which may prove to be allergic to the person. Furthermore existing methods require eye fixation i.e. person needs to be in steady position for a relatively long time.

Therefore, in light of the foregoing discussion, there exists a need to overcome the aforementioned drawbacks associated with existing measurement techniques for measuring properties of the eye.

### SUMMARY

The present disclosure seeks to provide an apparatus for measuring property of an eye. The present disclosure also seeks to provide a method for measuring property of an eye. An aim of the present disclosure is to provide a solution that overcomes at least partially the problems encountered in prior art.

In one aspect, the present disclosure provides an apparatus for measuring property of an eye, the apparatus comprising
an elongated magnetic probe having a probe head, wherein the probe head comprises an ultrasonic sensor therein;
a driver coil arranged partially to surround the elongated magnetic probe; and
a controller configured to, when in use
   selectively energize the driver coil to create a magnetic force to initiate movement of the elongated magnetic probe to contact the probe head with a surface of the eye, wherein the ultrasonic sensor in the probe head is configured to send an ultrasonic pulse upon contacting the surface of the eye;
   measure, using the ultrasonic sensor, time taken to receive at least one reflection of plurality of reflections of the ultrasonic pulse; and
   calculate the property of the eye using value of the time taken to receive at least one reflection of the ultrasonic pulse.

In another aspect, the present disclosure provides a method for measuring property of an eye, the method comprising
selectively energizing a driver coil to create a magnetic force to initiate movement of an elongated magnetic probe to contact a probe head with a surface of the eye, wherein the elongated magnetic probe comprises an ultrasonic sensor in the probe head thereof and the ultrasonic sensor is configured to send an ultrasonic pulse upon contacting the surface of the eye;
measuring, using the ultrasonic sensor, time taken to receive at least one reflection of a plurality of reflections of the ultrasonic pulse; and
calculating the property of the eye using value of the time taken to receive the at least one reflection of the ultrasonic pulse.

Embodiments of the present disclosure substantially eliminate or at least partially address the aforementioned problems in the prior art, and provides an efficient specialized equipment for measurement of the property of the eye.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those skilled in the art will understand that the drawings are not to scale. Wherever possible, like elements have been indicated by identical numbers.

Embodiments of the present disclosure will now be described, by way of example only, with reference to the following diagrams wherein:

FIG. 1 is a diagram of an apparatus, in accordance with an embodiment of the present disclosure;

FIG. 2 is a schematic illustration that illustrates different reflections from the eye upon contact of probe with surface of the eye, in accordance with an embodiment of the present disclosure;

FIG. 3 is a setup for measurement of property of an eye, in accordance with an embodiment of the present disclosure;

FIG. 4 is a graph to determine a period of time a probe head contacts the surface of eye using velocity profile and activate ultrasonic sensor during at least part of the period of time, in accordance with an implementation of the present disclosure;

FIG. 5 is a diagram of an apparatus comprising an elongated magnetic probe, in accordance with an embodiment of the present disclosure;

FIG. 6 is an apparatus for measuring property of an eye, in accordance with an embodiment of the present disclosure;

FIG. 7A is a front cross-sectional view and side cross-sectional view of Piezoelectric Micromachined Ultrasound Transducer (PMUT) element, in accordance with the embodiment of the present disclosure;

FIG. 7B is a cross-sectional view of Capacitive Micromachined Ultrasound Transducer (CMUT) element, in accordance with an embodiment of the present disclosure; and

FIG. 8 is a flow chart depicting steps of a method for measuring property of an eye, in accordance with an embodiment of the present disclosure.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practising the present disclosure are also possible.

In one aspect, the present disclosure provides an apparatus for measuring property of an eye, the apparatus comprising
an elongated magnetic probe having a probe head, wherein the probe head comprises an ultrasonic sensor therein;
a driver coil arranged partially to surround the elongated magnetic probe; and
a controller configured to, when in use
   selectively energize the driver coil to create a magnetic force to initiate movement of the elongated magnetic probe to contact the probe head with a surface of the eye, wherein the ultrasonic sensor in the probe head is configured to send an ultrasonic pulse upon contacting the surface of the eye;
   measure, using the ultrasonic sensor, time taken to receive at least one reflection of plurality of reflections of the ultrasonic pulse; and
   calculate the property of the eye using value of the time taken to receive at least one reflection of the ultrasonic pulse.

In another aspect, the present disclosure provides a method for measuring property of an eye, the method comprising
selectively energizing a driver coil to create a magnetic force to initiate movement of an elongated magnetic probe to contact a probe head with a surface of the eye, wherein the elongated magnetic probe comprises an ultrasonic sensor in the probe head thereof and the ultrasonic sensor is configured to send an ultrasonic pulse upon contacting the surface of the eye;
measuring, using the ultrasonic sensor, time taken to receive at least one reflection of a plurality of reflections of the ultrasonic pulse; and
calculating the property of the eye using value of the time taken to receive the at least one reflection of the ultrasonic pulse.

The apparatus and the method of the present disclosure aims to provide a measurement technique for efficient measurement of properties of the eye. Herein, the property of the eye, such as axial length is measured, wherein the apparatus takes a short measurement time and is not in contact with the surface of the eye for a prolonged duration of time. Furthermore, the apparatus requires no anaesthetic, as it bounces off the surface the eye as soon as the apparatus comes in contact with the eye. Typically, measuring the axial length of the eye enables investigation of myopia control strategies, and is increasingly important to reduce axial elongation to reduce lifelong health risk of the eye.

Throughout the present disclosure, the term “apparatus” refers to an instrument that is used to measure a property of an eye, wherein the property of the eye may be a physiological parameter concerning the eye such as, intra-ocular pressure of the eye, touch sensitivity and so forth. Furthermore, the intra-ocular pressure is calculated from a voltage signal representing velocity as a function of time of an elongated magnetic probe rebounded from surface of the eye. Typically, the magnitude of the voltage signal depends on the magnetic strength and speed of the elongated magnetic probe.

Optionally, the property of the eye is an axial length. Typically, the eye comprises an anterior segment that includes cornea of the eye, and posterior pole of the eye, which refers to retina between optic disc and macula. Herein, the term “axial length” refers to physical distance between the anterior segment and an arbitrary position at the posterior pole. Additionally, the axial length of the eye is a key parameter in determining status of refractive error. For instance, most significant structural aspect of myopia progression is growth of the axial length. Moreover, the eye grows extensively after birth. Additionally, the eye of a full-term new-born has a mean axial length ranging from 16 millimetres (mm) up to 18 mm & mean anterior chamber depth that may range from 1.5 mm up to 2.9 mm. The mean anterior chamber depth may for example be from 1.5, 1.8, 2.1, 2.5 or 2.8 mm up to 1.8, 2.1, 2.5, 2.8 or 2.9 mm. Furthermore, mean values for axial length for an adult may range from 22 mm up to 25 mm and mean refractive power for an adult may be in range of −25.0-+1.0 dioptres (D). Additionally, mean depth of anterior chamber in an emmetropic eye of an adult may be in the range from 3 mm up to 4 mm.

Optionally, the apparatus is rebound tonometer, wherein the rebound tonometer is used for measuring intra-ocular pressure (IOP) during ophthalmic measurement of the eye. Herein, the rebound tonometer processes rebound movement of a rod probe resulting from interaction of the rod probe with the eye. Furthermore, the rod probe may be a disposable rod probe comprising a magnetized steel wire shaft covered with a round plastic tip at the end that touches the eye, to minimize risk of corneal injury from impact of the rod probe. Typically, the rod probe hits the eye and bounces back upon switching on the rebound tonometer. Herein, the bouncing back of the rod probe depends on the intra-ocular pressure of the eye, wherein intra-ocular pressure is a measurement involving magnitude of force exerted by aqueous humor on internal surface area of the anterior segment of the eye. Moreover, the rod probe bounces faster as the intra-ocular pressure increases. Consequently, impact of the rod probe on the eye is shorter when value of the IOP is higher. Use of the rebound tonometer is beneficial as it enables short duration of contact time with the eye. During, said contact time the ultrasonic measurement can be carried out. Also usage of rebound tonometer is straightforward, and can be done easily also by non-doctors.

The apparatus comprises an elongated magnetic probe having a probe head, wherein the probe head comprises an ultrasonic sensor. Herein, the elongated magnetic probe has a first end, a second end opposite to the first end and a middle section between the first end and the second end. Notably, the first end of the elongated magnetic probe is made from bio-compatible material and will collide with surface of the eye when in use. Beneficially, the first part being made of bio-compatible material enables the probe to function in intimate contact with living tissues of the eye causing minimal discomfort or pain. Notably, the bio-compatible material is free from carcinogenicity, toxicity, and is resistive to corrosion. Furthermore, the elongated magnetic probe may be made of thin wire of magnetic material. Herein, the elongated magnetic probe may be for instance, 20 millimetres in length and 0.5 millimetre in width. Additionally, the magnetic material in the elongated magnetic probe may be ferromagnetic. Moreover, the movement of the elongated magnetic probe inside a measurement coil produces an induced voltage when it moves. However, the magnetic forces of the elongated magnetic probe are insignificantly small, wherein the elongated magnetic probe is pushed back only by the surface of the eye, with no other force being present. Notably, the elongated magnetic probe has a probe head. Additionally, the probe head enable touching the surface of the eye without any scarring. Furthermore, the probe head comprises an ultrasonic sensor.

Throughout the present disclosure, the term “ultrasonic sensor” refers to an instrument that measures distance to an object using ultrasonic sound waves. Additionally, the ultrasonic sensor uses a transducer to send and receive the ultrasonic sound waves that relays information about proximity of the object. Typically, high-frequency sound waves reflect from boundaries to produce distinct echo patterns. Furthermore, the ultrasonic sensors work by sending out an ultrasonic sound wave at a frequency above range of human hearing, wherein the range of human hearing is between 20 hertz to 20,000 hertz. Furthermore, the transducer of the ultrasonic sensor acts as a microphone to receive and send the ultrasonic sound waves. Additionally, the ultrasonic sensor determines distance to the object by measuring time lapses between the sending and receiving of the ultrasonic sound waves. For the sake of brevity, hereinafter the term “ultrasonic sound waves” is used interchangeably with the term “ultrasonic pulse”.

Optionally, the ultrasonic sensor comprises at least one of: a Piezoelectric Micromachined Ultrasound Transducer (PMUT) element coupled with an induction coil or Capacitive Micromachined Ultrasound Transducer (CMUT) element coupled with the induction coil. Herein, the PMUT element is a micro-electro-mechanical system (MEMS) based piezoelectric ultrasonic transducers. Notably, PMUTs are based on flexural motion of a thin membrane coupled with a thin piezoelectric film, such as polyvinylidene fluoride (PVDF). Additionally, PMUT has increased bandwidth, flexible geometries, natural acoustic impedance match with water, reduced voltage requirements, mixing of different resonant frequencies and potential for integration with supporting electronic circuits especially for miniaturized high frequency applications. Furthermore, the PMUTs are used for detection which allows the PMUTs to be integrated into applications such as biometric fingerprint and time-of-flight applications in the air, such as gesture recognition. Simultaneously, the CMUT element comprises transducers in which energy transduction occurs due to change in capacitance. Additionally, CMUTs are constructed on silicon using micromachining techniques. Herein, a cavity is formed in a silicon substrate, and a thin layer suspended on the top of the cavity serves as a membrane on which a metallized layer acts an electrode, together with the silicon substrate which serves as a bottom electrode. Notably, CMUTs allow excellent image resolution, which makes them candidates for medical imaging applications. Herein, “induction coil” refers to an electrical device for producing an intermittent source of high voltage. Additionally, the induction coil produces an electromagnetic field to transfer energy to a work piece to be heated. Consequently, as electrical current passes along a wire, a magnetic field is produced around that wire.

Optionally, in an example, the structure of the Piezoelectric Micromachined Ultrasound Transducer (PMUT) consists of a silicon membrane supported by a silicon structure. Additionally, a piezo material is located between two metal electrodes. Moreover, an alternating voltage between the electrodes generates a traverse stress and a bending moment resulting in the deflection of the membrane in phase with voltage. Consequently, acoustic waves are formed in the surrounding media.

Optionally, in another example, the structure of the Capacitive Micromachined Ultrasound Transducer (CMUT) consists of a silicon membrane supported by silicon oxide layer. Additionally, an electrode gap is created in between top metal electrodes and bottom metal electrode. Moreover, a voltage in between the top metal electrode and the bottom metal electrode produces an electrostatic force and the silicon membrane deflects. Furthermore, the silicon membrane starts to vibrate in phase with the voltage and generates acoustic waves in surrounding media when applying direct voltage and/or alternating voltage.

Optionally, the coupling to induction coil is used to provide energy for the ultrasonic sensor and/or provide communication link between the ultrasonic sensor and the controller. Herein, the induction coil produces electromagnetic field when an electric current is passed through the induction coil, and the electromagnetic field is produced around the induction coil. Notably, the induction coil is capable of generating high voltages. Additionally, the induction coil provides energy to the coupled ultrasonic sensor with Piezoelectric Micromachined Ultrasound Transducer (PMUT) or Capacitive Micromachined Ultrasound Transducer (CMUT) element. Moreover, the induction coil further acts as a communication link between the ultrasonic sensor and the controller. Herein, communication data may include measurement results.

The apparatus comprises a driver coil arranged partially to surround the elongated magnetic probe. Herein, the driver coil is arranged as a loop through which the elongated magnetic probe can move. Furthermore, the driver coil has finite number of loops. Additionally, the driver coil may be arranged along any point between the first end and the second end. For instance, the driver coil may be arranged closer to the first end. Subsequently, the driver coil moves the elongated magnetic probe when electric current is fed through the driver coil. Furthermore, the driver coil pulls the elongated magnetic probe and projected towards the surface of the eye with a velocity which is equal to product of the electric current fed through the driver coil times the elongated magnetic probe magnetization.

The apparatus comprises a controller. Herein, the controller is a computational device that is operable to respond to and process information. In an example, the controller may be an embedded microcontroller, a microprocessor, computer, or a portable computing device. Furthermore, the controller is communicably coupled with the measurement coil and the driver coil. Additionally, the controller energizes the driver coil to move the elongated magnetic probe towards the surface of the eye.

The controller is configured to selectively energize the driver coil to create a magnetic force to initiate movement of the elongated magnetic probe to contact the probe head with a surface of the eye, wherein the ultrasonic sensor in the probe head is configured to send an ultrasonic pulse upon contacting the surface of the eye. Herein, the term “selectively energize” refers to switching the supply voltage of the driver coil ON or OFF. Notably, an electric field is created when the supply voltage of the driver coil is switched ON. Typically, higher supply voltage of the driver coil will result in greater magnetic force. Subsequently, the acceleration of the elongated magnetic probe will increase. Moreover, the magnetic force is a function of magnetization of the elongated magnetic probe. Herein, the magnetization refers to a strength of magnetisation of the elongated magnetic probe. In particular, higher magnetization will result in greater magnetic force. Additionally, selectively energizing the driver coil will move the elongated magnetic probe towards the surface of the eye. Moreover, the elongated magnetic probe will move in an alternate direction in case polarity of the driver coil by selectively energizing is reversed. Herein, the elongated magnetic probe moves in a direction which may be controlled by the controller whenever required, wherein the voltage is a function of speed of the elongated magnetic probe and magnetization of the elongated magnetic probe. Additionally, the speed of the elongated magnetic probe is monitored continuously by the measurement coil as a function of time. Subsequently, such information relating to the speed of the elongated magnetic probe may be used for determining pressure of the eye which may be used for diagnostic purposes. Typically, a magnetic field is induced in the driver coil when the electric current is provided to the driver coil. Furthermore, the magnetic field is proportional to the electric current and the finite number of loops of the driver coil. Typically, the magnetic field can be controlled by controlling the electric current provided to the driver coil. Subsequently, the magnetic field causes the magnetic force on the elongated magnetic probe, wherein the magnetic force is a function of magnetization of the elongated magnetic probe. Furthermore, acceleration of the elongated magnetic probe is calculated by dividing mass of the elongated magnetic probe by the force on the elongated magnetic probe. Henceforth, the elongated magnetic probe is accelerated by current pulse in the driver coil, thereby generating a magnetic field acting on the elongated magnetic probe. Furthermore, the elongated magnetic probe is sent through the measurement coil towards the surface of the eye, from which the elongated magnetic probe bounces back. Notably, a magnetization cycle is integrated in the present disclosure for magnetization of the elongated magnetic probe. Herein, the magnetization cycle is achieved by current pulse in the driver coil and the measurement coil respectively, thereby pulling the elongated magnetic probe back and forth in the apparatus. Furthermore, the magnetization cycle may end with a calibration cycle. Herein, the calibration cycle collects the information required to set the acceleration in the driver coil to a value for obtaining an optimal speed for the elongated magnetic probe. Furthermore, the calibration cycle checks both the apparatus and the elongated magnetic probe, before determining the property of the eye. The initiated movement refers to ejecting the elongated magnetic probe towards eye. After initiating the movement the elongated magnetic probe will move “freely” without driving towards the eye and after contacting will bounce back from the eye without using driving force. This initiated movement, resulting to ejecting the elongated magnetic probe, thus allows short duration contact with the probe head and the eye. The short duration of the contact allows to use the apparatus without for example ocular anesthesia.

Optionally, the apparatus comprises a measurement coil configured to measure velocity profile of the elongated magnetic probe during the initiated movement of the elongated magnetic probe. Herein, the measurement coil comprises at least a first section and a second section, and the measurement coil arranged partially to surround the magnetic probe. Notably, the measurement coil has a finite number of loops. Furthermore, the total number of loops of the measurement coil are divided between the first section and the second section. Herein, each of the first section and the second section may have at least one loop through which the elongated magnetic probe is able to move. Furthermore, the elongated magnetic probe may move along the middle section, wherein the middle section is between the first section and the second section.

Optionally the initiated movement of the elongated magnetic probe is a rebound movement. The rebound movement comprises, when in use, a ejection the elongated magnetic probe towards they eye, a contact of the elongated magnetic probe with the eye and a bounce back for the elongated magnetic probe from the eye. Indeed the initiated movement refers to cycle of ejecting (thus accelerating the probe) towards the eye. After the acceleration the elongated magnetic probe will move freely i.e. without external force from the drive coil towards the eye. As the elongated magnetic probe contacts the eye it will start to deaccelerate due to force of eye surface. During said contact time the ultrasonic sensor is used to send ultrasonic pulse (or pulses) and measure time taken to receive at least one reflection. The elongated magnetic probe will eventually bounce back from the eye inside (or at least partly inside) of the driver coil of the apparatus. This completes one measurement cycle. The measurement cycle can be repeated. The rebound movement provide a gentle, quick touch to eye thus making the measurement pleasant for a patient. Optionally, in this regard, the measurement coil is used to drive the electric current, thereby inducing a motion to the elongated magnetic probe. Subsequently, immediately after the elongated magnetic probe is accelerated to its operational velocity, the measurement coil is used to measure induced voltage to the measurement coil. Beneficially, the number of coils in the apparatus is reduced. Furthermore, the measurement coil may have multiple functions depending on the manner a controller controls the measurement coil, such as for example whether electric current is driven, or induced voltage is measured.

Optionally, in this regard, the first section and the second section of the measurement coil are connected in series. Herein, series connection refers to an electrical coupling of the first loop of the first section to the first loop of the second section. Typically, a series connection is realized by connecting a voltmeter to the first section of the measurement coil and the first section of the driver coil. Alternatively, the induced voltage over the first section is measured. Subsequently, the induced voltage over both the first section and the second section may be measured together.

Optionally, in this regard, the measurement coil comprises a third section, the third section connected in series with the first and the second section of the measurement coil and the third section is arranged to surround a third section of the elongated magnetic probe. Herein, the third section may have at least one loop through which the elongated magnetic probe is able to move. Furthermore, multiple sections may be added in the measurement coil, thereby generating more data points.

Optionally, in this regard, the velocity profile of the elongated magnetic probe is calculated to determine the property of the eye. Moreover, depending on target application, velocity as function of time is used to determine the property of the eye i.e., the physiological parameter, such as for example, the IOP. In an example, the speed of the elongated magnetic probe and response of the surface of the eye to stop the elongated magnetic probe once it is ejected towards the surface of the eye may be determined by calculating first derivate of the velocity. Furthermore, the speed of the elongated magnetic probe rebounding from the surface of the eye may be determined. Herein, the IOP is high in case the elongated magnetic probe rebounds rapidly, and the IOP is low in case the elongated magnetic probe rebounds slowly. Subsequently, the measurements regarding the time and velocity of the elongated magnetic probe may be collected with medical trials and function of speed of the elongated magnetic probe form factors is compared with medical trial measurements as discussed in the present disclosure to determine physiological parameters. Furthermore, the speeds of the elongated magnetic probe may be determined by dividing the first induced voltage value to the second induced voltage value, thereby giving a relative value which is used to determine the speed of the elongated magnetic probe.

In an example, the measurement coil is divided into the first section, the second section, the third section and the fourth section, and the driver coil comprises one section. Herein, a plurality of coupling leads are wired to the points connecting the first section, the second section, the third section and the fourth section of the measurement coil to each other. Furthermore, a first coupling lead is connected to the leftmost end of the first section, a second coupling lead is connected to the junction of the first section and the second section, a third coupling lead is connected to the junction of the second section and the third section, a fourth coupling lead is connected to the junction of the third section and the fourth section, and a fifth coupling lead is connected to the rightmost end of the fourth section.

Optionally, the ultrasonic sensor is activated when a first derivate in respect to time of the velocity profile is below pre-set threshold value. Herein, the first derivate in respect to the time of the velocity profile relates to acceleration or deceleration of the probe head. Typically, the probe head is ejected towards the surface of the eye. Subsequently, the ultrasonic sensor is activated as soon as the probe head touches the surface of the eye i.e., the cornea and starts to decelerate after reaching a pre-set threshold value, thereby making the probe head bounce back. Alternatively the first derivate of the velocity profile can be used to find a moment of time on which the direction of the ultrasonic sensor changes (from going forward towards eye and bouncing back i.e. derivate is zero). During this moment a touching force between sensor and the eye is largest. According to one embodiment the ultrasonic sensor is activated at said moment of time. Alternatively the activation can take place for example at moment of time which is half way between the touching they eye and the derivate being zero.

The controller is configured to measure, using the ultrasonic sensor, time taken to receive at least one reflection of plurality of reflections of the ultrasonic pulse. Notably, the probe head touches the surface of the eye and ultrasonic measurements are carried out by the ultrasonic sensor during time of contact. Subsequently, the ultrasonic sensor starts sending ultrasonic pulses as the probe head touches the eye. Moreover, the time taken to receive the reflection of the ultrasonic pulses upon return is determined as the ultrasonic pulses travel in the eye. Furthermore, the measurement may be performed multiple times, such as for example, but not limited to, 2, 4, 5, 10 and so forth times to procure average values of the time taken to receive the at least one reflection of the plurality of reflections of the ultrasonic pulse. Notably, the time taken for the at least one reflection of the plurality of reflections of the ultrasonic pulse are used to calculate the property of the eye, such as, the axial length of the eye. As an example, since speed of sound in eye (consisting mostly water) is about v=1640 m/sec, time taken for a reflection can be used to calculate the axial length. As an example if a reflection of ultrasonic pulse takes 23 microsec it corresponds to ½×1640 m/sec×23 usec=19 mm of axial length. Other properties (such as structures of inner parts of the eye) can be also calculated by looking on duration of different pulse reflections from different layers of the eye.

Optionally, the controller is configured to measure, using the ultrasonic sensor, from the plurality of reflections time taken to receive reflections of the ultrasonic pulse corresponding to interfaces between parts of the eye. Herein, the ultrasonic sensor uses echo, or a brightness scan (B-scan) to assess structural integrity and pathology of the eye by producing a two-dimensional cross-section of the eye and orbit of the eye. Furthermore, the plurality of reflections are taken from refracting surfaces of anterior (Cₐ) and posterior (Cₚ) segments of the cornea, refracting surfaces of anterior (Lₐ) and posterior (Lₚ) segments of the lens, the retina (R) and sclera (S) of the eye.

Optionally, the controller is further configured to measure, using the ultrasonic sensor, amplitude of the plurality of received reflections of the ultrasonic pulse and use the measures amplitudes to select the at least one reflection. Herein, the ultrasonic sensor uses amplitude scan (A-scan) to provide details about the axial length of the eye. Furthermore, the A-scan provides a one-dimensional scan of the eye and determines the axial length of the eye for calculation of intraocular lens power. Additionally, reading of the A-scan ideally shows five spikes with each echo rise 90 degrees to baseline.

The controller is configured to calculate the property of the eye using value of the time taken to receive at least one reflection of the ultrasonic pulse. Herein, the cornea of the eye may have an average central corneal thickness (CCT) of 540 micrometres (μm). Furthermore, the CCT may possess a normal range from 480 μm up to 570 μm. The normal range of the CCT may for example be from 480, 500, 520, 540 or 560 μm up to 500, 520, 540, 560 or 570 μm. Additionally, speed of the sound waves in cornea of the eye may be 1640 metres per second. Theoretically, bandwidth of the ultrasonic pulse may be at least 2 megahertz or higher. Subsequently, the time of contact of the probe head with the surface of the eye may be 2 milliseconds. Time duration of a contact depends on the velocity of the elongated magnetic probe and a pressure of the eye. The contact time duration can be for example 1 milliseconds to 4 milliseconds for example 1 to 3 milliseconds or 1.5 to 2.5 milliseconds. Therefore, resolution of the eye may be calculated, thereby providing the axial length of the eye. I.e., according to an embodiment a set of measurement pulses can be sent during the time of contact. This can be used to calculate average values etc to improve signal to noise ratio.

Optionally, the controller is configured to determine a period of time the probe head contacts the surface of the eye using the velocity profile and activate the ultrasonic sensor during the at least part of the period of time. Herein, the ultrasonic sensor located in the probe head sends ultrasonic pulse as soon as it contacts the surface of the eye. Additionally, the ultrasonic sensor monitors movement of the sensor to detect when the sensor is in contact with the eye. Alternatively, the ultrasonic sensor may be ON all the time and only the pulses in which the reflection is seen (i.e., when the ultrasonic sensor touches the eye) are used. Furthermore, the ultrasonic sensor may be turned ON (i.e. activated) when it is fully in contact with the eye. Moreover, the full contact may be determined by the principle at the time when velocity of the probe head approaches zero. The present disclosure also relates to the method as described above. Various embodiments and variants disclosed above apply mutatis mutandis to the method.

Optionally, the method comprises measuring, using the ultrasonic sensor, from the plurality of reflections time taken to receive reflections of the ultrasonic pulse corresponding to interfaces between parts of the eye.

Optionally, the method comprises measuring, using the ultrasonic sensor, amplitude of the plurality of received reflections of the ultrasonic pulse and using the measured amplitudes for selecting the least one reflection.

Optionally, a velocity profile of the elongated magnetic probe is measured during the initiated movement of the elongated magnetic probe.

Optionally, the method comprises determining a period of time the probe head is contacting the surface of the eye using the velocity profile and activating the ultrasonic sensor for a at least part of the period of time.

Optionally, the ultrasonic sensor is activated when a first derivate in respect to time of the velocity profile is below preset threshold value.

Optionally, the property of an eye is axial length of the eye.

Optionally or additionally it is possible to perform repetitively a plurality of measurements by ultrasonic, to detect variations in the anatomic dimensions (anatomic properties) over time (such as over week, month, year etc), and which variations could give valuable information for example if there is for example some disease developing in the examined eye. This way the apparatus can be used to provide trends for patient (user) related to how certain property of eye develops over time.

The present disclosure further provides an elongated magnetic probe having a probe head, wherein the probe head comprises an ultrasonic sensor therein.

Optionally, the elongated magnetic probe further comprises an induction arrangement for receiving energy and to provide a communication link.

### DETAILED DESCRIPTION OF THE DRAWINGS

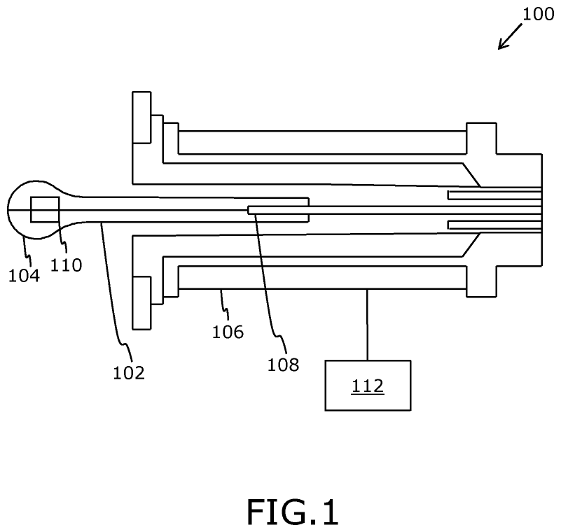

Referring to FIG. 1, there is shown a diagram of an apparatus 100, in accordance with an embodiment of the present disclosure. Herein, the apparatus 100 comprises elongated magnetic probe 102, wherein the elongated magnetic probe 102 further comprises a probe head 104. Furthermore, the apparatus 100 comprises a driver coil 106 arranged partially to surround the elongated magnetic probe 102. Additionally, the elongated magnetic probe 102 may be made of thin wire 108 of magnetic material. The probe head 104 comprises an ultrasonic sensor 110. The apparatus 100 further comprises a controller 112 to selectively energize the driver coil 106 to create a magnetic force to initiate movement of the elongated magnetic probe 102 to contact the probe head 104 with a surface of an eye. An example of the initiated movement is a rebound movement. In the rebound movement the initiated movement starts by ejection of the elongated magnetic probe towards the surface of the eye. The elongated magnetic probe will collide with the eye surface and will be in contact with the eye before bouncing back from the eye. Additionally the driver coil might be, after using it to eject the elongated magnetic probe towards eye to measure induced magnetic field resulting from movement of the elongated magnetic probe. The measured induced magnetic field can be used to determine velocity profile of the elongated magnetic probe.

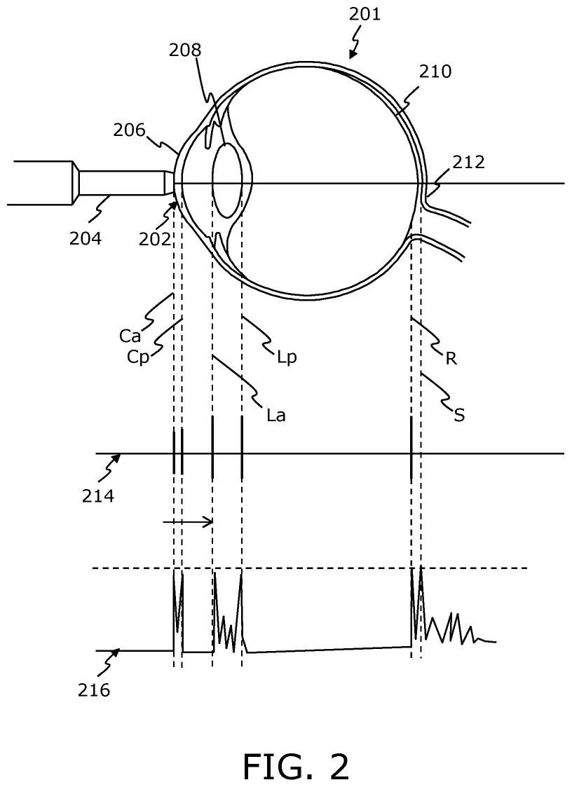

Referring to FIG. 2, there is shown a schematic illustration that illustrates different reflections from the eye 201 upon contact of probe with surface of the eye 202, in accordance with an embodiment of the present disclosure. Herein, a probe head 204 touches the surface of the eye 202. The probe head 204 comprises an ultrasonic sensor to measure plurality of reflections, time taken to receive reflections of the ultrasonic pulse corresponding to interfaces between parts of the eye. Herein, the plurality of reflections are taken from refracting surfaces of anterior (Cₐ) and posterior (Cₚ) segments of the cornea 206, refracting surfaces of anterior (Lₐ) and posterior (Lₚ) segments of the lens 208, the retina (R) 210 and sclera (S) 212 of the eye 202. Furthermore, graph 214 depicts reduced eye optics with relation to amplitude scan (A-scan) structures. Additionally, graph 216 depicts an example A-scan reading that shows several spikes, wherein with each echo rises 90 degrees to baseline.

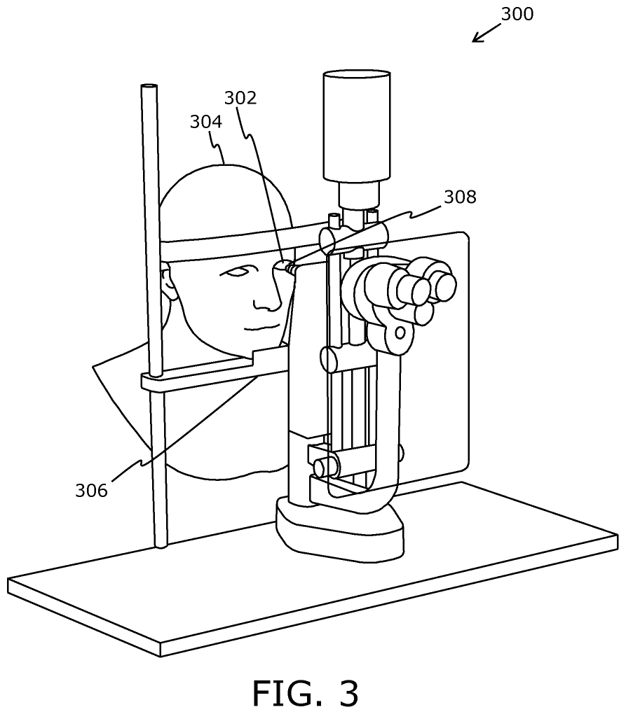

Referring to FIG. 3, there is shown a setup 300 for measurement of property of an eye 302, in accordance with an embodiment of the present disclosure. Herein, a patient 304 sits upright, wherein the patient 304 places chin on a chin rest 306 and looks straight ahead. Subsequently, when device is used a probe head 308 comes in contact with surface of the eye as the eye measured using an ultrasonic pulse

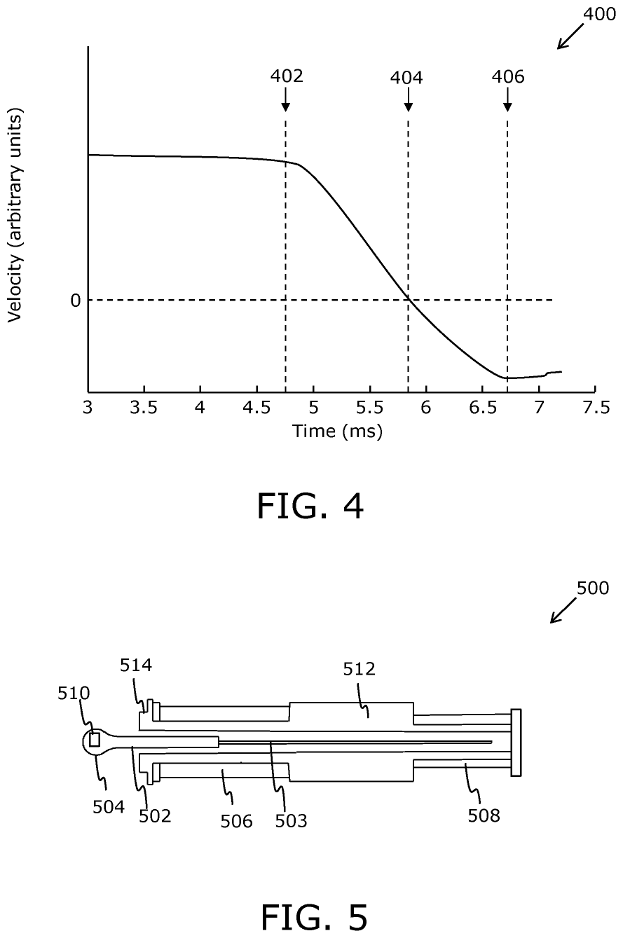

Referring to FIG. 4, there is shown a graph 400 to determine a period of time a probe head contacts the surface of eye using velocity profile and activate ultrasonic sensor during at least part of the period of time, in accordance with an implementation of the present disclosure. The horizontal axis represents time in millisecond (ms) of the movement of the probe head. The vertical axis represents velocity profile of the probe head in arbitrary units, s). Herein, line 402 depicts that the instance when probe head touches cornea of the eye. Furthermore, line 404 depicts the instance when the cornea is flattened by the probe head. Herein, a first derivate in respect to time of the velocity profile decelerates after reaching a pre-set threshold value. Additionally, line 406 depicts the instance when the probe head bounces back after reaching the pre-set threshold value. According to an embodiment the ultrasonic measurement is carried out between time starting at 402 and ending at 406 (i.e. about between time of 4.7 ms (millisecond) to 6.7 ms resulting to a contact time of 2 milliseconds). During said time one can perform at least one measurement. Depending on the setup one can perform up to 10, 100 or 1000 measurements during the said time of contact (about 2 msec). This is beneficial as several measurements can be used to derive better signal to noise ratio in comparison to a one measurement. Further, as the probe is ejected towards the eye several times the measurements can be repeated. Short contact time of about 2 milliseconds is significantly shorter than reaction time of eye (which is typically in range of 200-300 msec) thus the measurement can be carried out before the person blinks the eye.

Referring to FIG. 5, there is shown a diagram of an apparatus 500 comprising an elongated magnetic probe 502, in accordance with an embodiment of the present disclosure. The elongated magnetic probe 502 further comprises a probe head 504. Ultrasonic sensor 506 is embedded in the probe head 504. Furthermore, the apparatus 500 comprises a driver coil 506 arranged partially to surround the elongated magnetic probe 502. Additionally, the apparatus 502 comprises a measurement coil 508 configured to measure velocity profile of the elongated magnetic probe 502 during initiated movement of the elongated magnetic probe 502. Moreover, the elongated magnetic probe 502 may be made of thin wire 510 of magnetic material. The elongated magnetic probe 502 is encased in a coil frame 512. The coil frame 512 comprises a probe base 514.

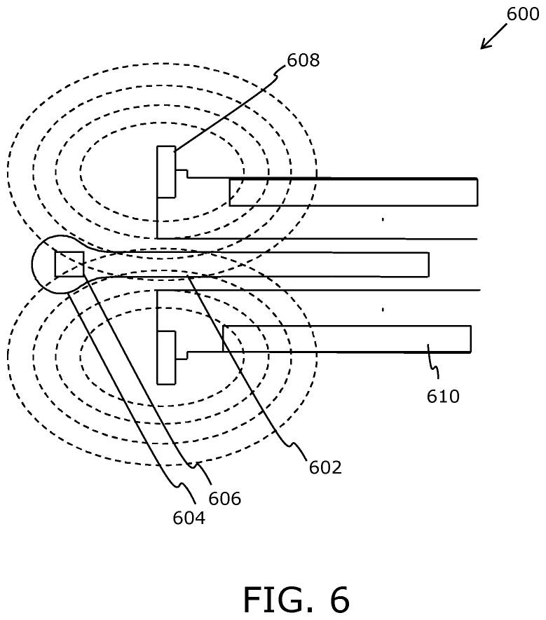

Referring to FIG. 6, there is shown an apparatus 600 for measuring property of an eye, in accordance with an embodiment of the present disclosure. Herein, the apparatus 600 comprises an elongated magnetic probe 602 having a probe head 604, wherein the probe head 604 comprises an ultrasonic sensor 606. Additionally, the ultrasonic sensor 606 comprises energy harvesting means coupled with the induction coil 608 to energize the ultrasonic sensor as well as transfer information to/from the ultrasonic sensor Induced magnetic field is illustrated with dashed lines. The magnetic field is used to energize/provide energy to the probe. The apparatus 600 comprises a driver coil 610 arranged partially to surround the elongated magnetic probe 602.

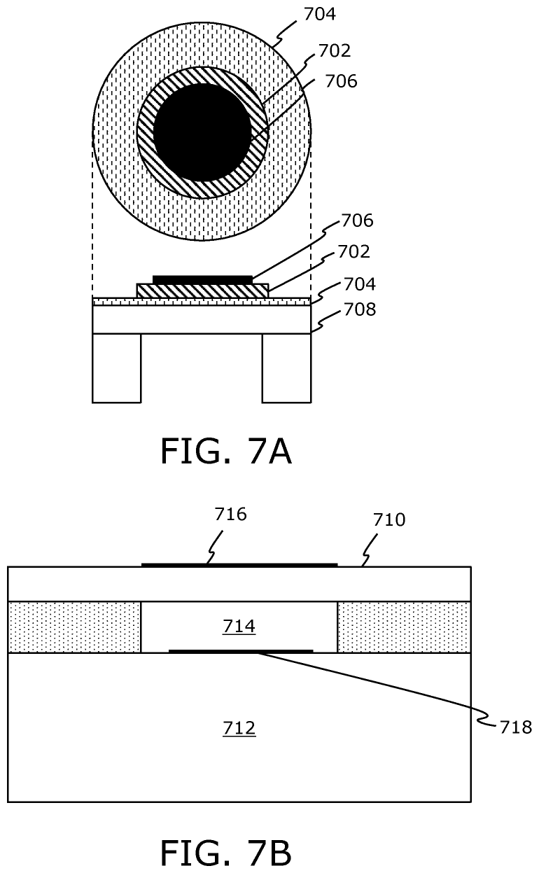

Referring to FIG. 7A, there is shown a front cross-sectional view and side cross-sectional view of Piezoelectric Micromachined Ultrasound Transducer (PMUT) element, in accordance with the embodiment of the present disclosure. The PMUT element comprises a piezo material 702 in middle of a first metal electrode 704 and a second metal electrode 706, with a base of silicon 708.

Referring to FIG. 7B, there is shown a cross-sectional view of Capacitive Micromachined Ultrasound Transducer (CMUT) element, in accordance with an embodiment of the present disclosure. Herein, the CMUT element consists a silicon membrane 710 supported by Silicon oxide substrate 712. Furthermore, the CMUT element comprises an electrode gap 714 in-between top metal electrode 716 and bottom metal electrode 718.

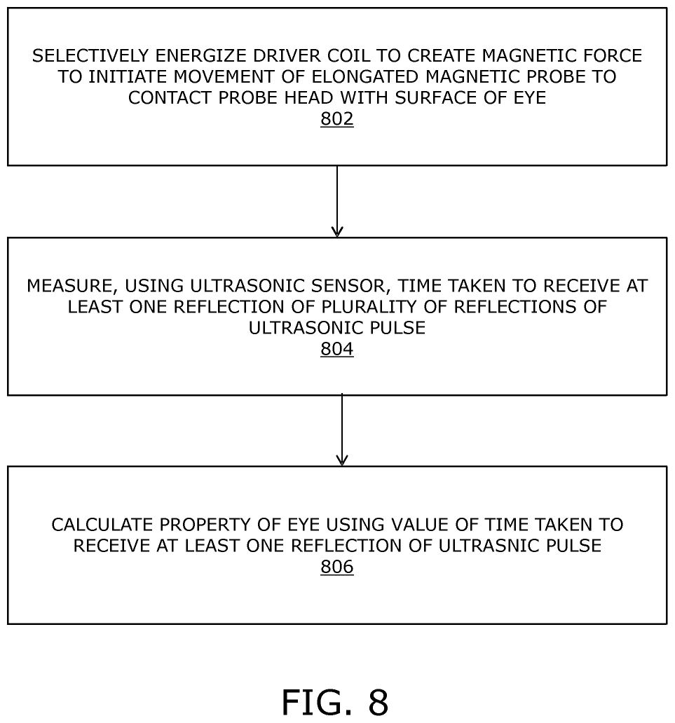

Referring to FIG. 8, there is shown a flow chart depicting steps of a method for measuring property of an eye, in accordance with an embodiment of the present disclosure. At step 802, a driver coil is selectively energized to create a magnetic force to initiate movement of an elongated magnetic probe to contact a probe head with a surface of the eye, wherein the elongated magnetic probe comprises an ultrasonic sensor in the probe head thereof and the ultrasonic sensor is configured to send an ultrasonic pulse upon contacting the surface of the eye. At step 804, time taken to receive at least one reflection of a plurality of reflections of the ultrasonic pulse is measured, using the ultrasonic sensor. At step 806, the property of the eye is calculated using value of the time taken to receive the at least one reflection of the ultrasonic pulse.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural.

1. An apparatus for measuring property of an eye, the apparatus comprising
an elongated magnetic probe having a probe head, wherein the probe head comprises an ultrasonic sensor therein;
a driver coil arranged partially to surround the elongated magnetic probe; and
a controller configured to, when in use
selectively energize the driver coil to create a magnetic force to initiate movement of the elongated magnetic probe to contact the probe head with a surface of the eye, wherein the ultrasonic sensor in the probe head is configured to send an ultrasonic pulse upon contacting the surface of the eye;
measure, using the ultrasonic sensor, time taken to receive at least one reflection of plurality of reflections of the ultrasonic pulse; and
calculate the property of the eye using value of the time taken to receive at least one reflection of the ultrasonic pulse.

2. The apparatus according to claim 1, wherein the controller is configured to measure, using the ultrasonic sensor, from the plurality of reflections time taken to receive reflections of the ultrasonic pulse corresponding to interfaces between parts of the eye.

3. The apparatus according to claim 1, wherein the controller is further configured to measure, using the ultrasonic sensor, amplitude of the plurality of received reflections of the ultrasonic pulse and use the measured amplitudes to select the least one reflection.

4. The apparatus according to claim 1, wherein the ultrasonic sensor comprises at least one of: a Piezoelectric Micromachined Ultrasound Transducer (PMUT) element coupled with an induction coil (608) or Capacitive Micromachined Ultrasound Transducer (CMUT) element coupled with the induction coil.

5. The apparatus according to claim 4, wherein the coupling to induction coil is used to provide energy for the ultrasonic sensor and/or provide communication link between the ultrasonic sensor and the controller.

6. The apparatus according to claim 4, further comprising a measurement coil configured to measure velocity profile of the elongated magnetic probe during the initiated movement of the elongated magnetic probe.

7. The apparatus according to claim 6, wherein the controller is configured to determine a period of time the probe head contacts the surface of the eye using the velocity profile and activate the ultrasonic sensor during at least part of the period of time.

8. The apparatus according to claim 7, wherein the ultrasonic sensor is activated when a first derivate in respect to time of the velocity profile is below preset threshold value.

9. The apparatus according to claim 7, wherein the initiated movement of the elongated magnetic probe is a rebound movement, the rebound movement comprising, when in use, an ejection of the elongated magnetic probe towards the eye, a contact of the elongated magnetic probe with the eye and a bounce back for the elongated magnetic probe from the eye.

10. The apparatus according to claim 7, wherein the apparatus is rebound tonometer.

11. The apparatus according to claim 7, wherein the property of the eye is an axial length.

12. A method for measuring property of an eye, the method comprising
selectively energizing a driver coil to create a magnetic force to initiate movement of an elongated magnetic probe to contact a probe head with a surface of the eye, wherein the elongated magnetic probe comprises an ultrasonic sensor in the probe head thereof and the ultrasonic sensor is configured to send an ultrasonic pulse upon contacting the surface of the eye;
measuring, using the ultrasonic sensor, time taken to receive at least one reflection of a plurality of reflections of the ultrasonic pulse; and
calculating the property of the eye using value of the time taken to receive the at least one reflection of the ultrasonic pulse.

13. The method according to claim 12, wherein the method comprises measuring, using the ultrasonic sensor, from the plurality of reflections time taken to receive reflections of the ultrasonic pulse corresponding to interfaces between parts of the eye.

14. The method according to claim 12, wherein the method further comprises measuring, using the ultrasonic sensor, amplitude of the plurality of received reflections of the ultrasonic pulse and using the measured amplitudes for selecting the least one reflection.

15. The method according to claim 12, further comprising measuring a velocity profile of the elongated magnetic probe during the initiated movement of the elongated magnetic probe.

16. The method according to claim 15, wherein the method comprises determining a period of time the probe head is contacting the surface of the eye using the velocity profile and activating the ultrasonic sensor for a at least part of the period of time.

17. The method according to claim 16, wherein the ultrasonic sensor is activated when a first derivate in respect to time of the velocity profile is below preset threshold value.

18. The method according to claim 12 wherein the property of an eye is axial length of the eye.

19. An elongated magnetic probe having a probe head, wherein the probe head comprises an ultrasonic sensor therein.

20. The elongated magnetic probe according to claim 19, wherein the elongated magnetic probe further comprises an induction arrangement for receiving energy and to provide a communication link.
